(19) United States
(12) Patent Application Publication
(10) Pub. No.: US 2009/0275853 A1
(43) Pub. Date: Nov. 5, 2009

(54) METHOD AND APPARATUS FOR MONITORING PHYSIOLOGICAL STATE OF A SUBJECT

(75) Inventor: Mika Sarkela, Espoo (FI)

Correspondence Address: Andrus, Sceales, Starke & Sawall, LLP, 100 East Wisconsin Avenue, Suite 1100, Milwaukee, WI, 53202-4178, US

(73) Assignee: THE GENERAL ELECTRIC COMPANY, Schenectady, NY (US)

(21) Appl. No.: 12/112,147

(22) Filed: Apr. 30, 2008

(51) Int. Cl. A61B 5/0476

(52) U.S. Cl. 600/544

(57) ABSTRACT

A method and apparatus for monitoring physiological state of a subject is disclosed. Physiological signal data obtained from a subject is decomposed into a plurality of signal subentities, such as subbands of the overall frequency band of the signal data. A first measure indicative of the entropy of the respective signal subentity is determined for each of the subentities, thereby to obtain a corresponding plurality of first measures. An aggregate entropy measure is then calculated from the plurality of first measures and the aggregate entropy measure is employed to produce at least one state index indicative of a physiological state of the subject. The aggregate entropy measure typically represents a sum of the plurality of first measures.

### BACKGROUND OF THE INVENTION

This disclosure relates generally to monitoring and analysis of the physiological state of a subject. The physiological state here refers to the physiological status of the subject or a particular organ of the subject, where the term physiological relates to physiology, the science dealing with the functions of living matter and beings. More particularly, this disclosure involves monitoring and analysis of a physiological signal, typically brain wave signal, based on the entropy measured from the signal.

Electroencephalography (EEG) is a well-established method for assessing brain activity. When measurement electrodes are attached on the skin of the skull surface, the weak biopotential signals generated in the pyramid cells of the cortex may be recorded and analyzed. The EEG has been in wide use for decades in basic research of the neural systems of the brain as well as in the clinical diagnosis of various central nervous system diseases and disorders.

One of the special applications of the EEG, which has received attention recently, is the use of a processed EEG signal for objective quantification of the amount and type of brain activity for the purpose of determining the level of consciousness of a subject. In its simplest form, the utilization of an EEG signal allows the automatic detection of the alertness of an individual, i.e., if he or she is awake or asleep. This has become an issue of increased interest, both scientifically and commercially, in the context of measuring the depth of hypnosis induced by anesthesia during surgery.

The depth of hypnosis is not directly measurable. Therefore, drug delivery systems have to derive the level of hypnosis from a surrogate signal or from variables derived from that signal. The most common and popular surrogate signal for this purpose is the EEG, from which several variables or parameters may be determined. The basic reason for the insufficiency of a single variable or parameter is the variety of drugs and the complexity of the drug effects on the EEG signal in human brains. However, during the past few years, some commercially validated devices for measuring the level of consciousness and/or awareness in clinical set-up during anesthesia or sedation have become available. These devices, which are based on a processed EEG signal, have been introduced e.g. by GE Healthcare Finland Oy, Kuortaneenkatu 2, FIN-00510 Helsinki (Entropy®) and by Aspect Medical Systems, Inc., One Upland Road, Norwood, Mass. 02062, U.S.A. Bispectral Index™ (BIS™) is a trademark of Aspect Medical Systems, Inc.

The Bispectral Index™ involves the calculation of three subparameters, BetaRatio, SyncFastSlow, and Burst Suppression, and the resulting index is a combination of the three subparameters. Some of the techniques for analyzing EEG signals in an effort to determine the depth of anesthesia as well as the principles of the Bispectral Index algorithm are described in Ira J. Rampil, A Primer for EEG Signal Processing in Anesthesia, Anesthesiology, Vol. 89(4) October 1998, pp. 980-1002.

In the S/5 Entropy Module of GE Healthcare Finland Oy, two spectral entropy variables termed State Entropy (SE) and Response Entropy (RE) are computed. State Entropy, which primarily reflects the cortical state of the subject, is computed over a frequency range from 0.8 Hz to 32 Hz, which corresponds to the EEG-dominant part of the spectrum. The Response Entropy, in turn, is computed over a frequency range from 0.8 Hz to 47 Hz, which also contains EMG frequencies. The difference between the State Entropy and the Response Entropy is then indicative of the EMG activation. A combined indication provided by the State Entropy and the said entropy difference is then used to assess the level of hypnosis or sedation. The S/5 Entropy Module is based on the mechanisms described in U.S. Pat. No. 6,801,803. The entropy calculation algorithm of the S/5 Entropy Module has also been described in Viertiö-Oja H, Maja V, Särkelä M, Talja P, Tenkanen N, Tolvanen-Laakso H, Paloheimo M, Vakkuri A, Yli-Hankala A, Meriläinen P: Description of the Entropy algorithm as applied in the Datex-Ohmeda S/5 Entropy Module, Acta Anaesthesiologica Scandinavica 2004; Volume 48: Issue 2: 154-161, 2004.

BIS Index and the above-mentioned spectral entropy variables are thus commonly perceived as measures of the hypnotic component of anesthesia, and the above-mentioned commercially validated devices perform generally well, especially in monitoring the changes in the level of hypnosis of an individual subject. However, the operation of the devices is not completely consistent for the different drugs that may be administered, but the BIS Index or SE/RE values at which the subjects lose or recover their consciousness depend on the drugs administered. This is basically due to the fact that different drugs affect the level of hypnosis through different mechanisms, which leads to drug dependent LOC (loss of consciousness) and ROC (recovery of consciousness) values.

It has also been suggested that the monitoring process that determines the measure of the level of hypnosis may be controlled in dependence on the drug administration data that describes desired features of the current drug administration process. In this way, the inconsistencies that a varying drug combination may cause in the measure may be decreased or eliminated so that the value remains substantially consistent regardless of the combination of drugs administered to the patient.

In addition to the drug-related inconsistency, the said measures of the level of hypnosis are also subject to inter-individual variation. Among other individual characteristics between subjects, the skull acts as a low-pass filter, and the filtering effect depends on the thickness of the skull. Therefore, inter-individual variation also causes inconsistency in the measured values.

Furthermore, eye movement artifacts are deleterious for the traditional measurement of the level of hypnosis. The algorithms of the above-mentioned commercially validated devices are therefore provided with built-in eye movement artifact detection. However, artifact detection may make the algorithms slower in detection of changes in the level of hypnosis.

Other EEG-based monitoring applications include monitoring of natural sleep and epilepsy, for example.

### BRIEF DESCRIPTION OF THE INVENTION

The above-mentioned shortcomings, disadvantages and problems are addressed herein which will be understood by reading and understanding the following specification.

The embodiments of the invention rest on the discovery that entropy as a physical concept is typically considered as an additive property and but an improperly applied entropy determination is not able to take this additivity into account. Entropy is typically calculated from an appropriate distribution using the well-known Shannon's entropy equation. In case of an analysis of a physiological signal, it is particularly important that this distribution is employed in a proper manner so that the special nature of the signal does not result in the above-discussed drawbacks.

For example, subbands of a physiological signal may be indicative of different phenomena, without any significant connection between the subbands. This is exactly the case when brain wave signals are concerned. In other words, the entropy over the entire EEG frequency range is not equal to the sum of the entropies of the consecutive subbands covering the said entire frequency range.

In the embodiments of the invention, an estimate of the total entropy of a physiological system, which is represented by a measured physiological signal, is calculated as an aggregate measure of the entropies of signal subentities. Although the aggregate measure typically represents a sum of the entropies of the signal subentities, any combined measure acting similarly as the sum may be utilized. The subentities here refer to the components into which the physiological signal may be decomposed, i.e., each subentity forms a defined part of the original signal. Furthermore, the number of the subentities is typically chosen so that the subentities cover substantially the entire frequency range of the physiological signal. In information theory, entropy refers to a measure that is indicative of the information content of a random set of variables. As a physical concept, entropy describes the level disorder within the system. In signal analysis, entropy is used to characterize irregularity, complexity, and/or unpredictability of the signal. In this context, entropy may involve any of the above characteristics. Although a subband of the signal (i.e., the signal components on the subband) typically represents a subentity, generally speaking the subentity is specific to the physiological signal in question. Instead of a set of subbands, the subentities may also comprise a set of signal waveforms, for example. The aggregate measure may be calculated as a weighted or unweighted sum of the subentity entropies. The weights may correspond, for example, to the probabilities of each subentity, thus following the foundations of information theory and physics.

In an embodiment, a method for monitoring the physiological state of a subject comprises obtaining physiological signal data from the subject, decomposing the physiological signal data into a plurality of signal subentities, and determining, for each of the signal subentities, a first measure indicative of the entropy of respective signal subentity, thereby to obtain a corresponding plurality of first measures. The method further comprises calculating an aggregate entropy measure from the plurality of first measures, the aggregate entropy measure being indicative of total entropy of the physiological signal data, and employing the aggregate entropy measure to produce at least one state index indicative of the physiological state of the subject.

In another embodiment, an apparatus for monitoring the physiological state of a subject comprises a signal decomposer configured to decompose physiological signal data obtained from the subject into a plurality of signal subentities and a determination unit configured to determine, for each of the signal subentities, a first measure indicative of the entropy of respective signal subentity, thereby to obtain a corresponding plurality of first measures. The apparatus further comprises a first calculation unit configured to calculate an aggregate entropy measure from the plurality of first measures, the aggregate entropy measure being indicative of total entropy of the physiological signal data and a second calculation unit configured to employ the aggregate entropy measure to produce at least one state index indicative of the physiological state of the subject.

The embodiments of the invention enable the determination of an entropy-based state index that behaves more consistently in connection with varying drug combinations without a need to use additional control devices for ascertaining that the state index is consistent with the current drug combination. Furthermore, the effect of interindividual variation on the entropy-based state index may be reduced. This applies both to subjects in drug-induced hypnosis and to subjects in natural sleep, and also to subjects with epileptiform activity in the EEG signal.

In yet another embodiment, a computer program product for an apparatus monitoring a subject comprises a first program code portion configured to decompose physiological signal data obtained from a subject into a plurality of signal subentities and a second program code portion configured to determine, for each of the signal subentities, a first measure indicative of the entropy of respective signal subentity, thereby to obtain a corresponding plurality of first measures. The computer program product further comprises a third program code portion configured to calculate an aggregate entropy measure from the plurality of first measures, the aggregate entropy measure being indicative of total entropy of the physiological signal data, and a fourth program code portion configured to employ the aggregate entropy measure to produce at least one state index indicative of a physiological state of the subject.

Various other features, objects, and advantages of the invention will be made apparent to those skilled in the art from the following detailed description and accompanying drawings.

### BRIEF DESCRIPTION OF THE DRAWINGS

FIG. 1 is a schematic diagram illustrating the determination of a state index indicative of the physiological state of the subject;

FIGS. 2a to 2c illustrate an example of traditional entropy values and entropy values calculated as the sum of subband entropies;

FIGS. 3a to 3c illustrate the behavior of various entropy calculation methods in connection with eye movement artifacts;

FIG. 4 illustrates one embodiment of the system according to the invention;

FIG. 5 illustrates the processing units of the control unit of FIG. 4;

FIG. 6 illustrates an embodiment in which the signal subentities correspond to scales of wavelet transform; and

FIGS. 7a and 7b illustrate an example of the behavior of the sum of subentity entropies in connection with an EEG signal including epileptiform activity.

### DETAILED DESCRIPTION OF THE INVENTION

A typical monitoring device of the invention produces, based on the entropy of the physiological signal data obtained from a subject, at least one measure of the relevant physiological state of a subject. Below, the said measure is termed a state index. Although the state index is typically indicative of the depth of hypnosis or sedation and thus also of the overall state of the subject, it may also be indicative of the physiological state of a particular organ of the subject. The physiological state may thus also refer to the physiological state of a particular organ.

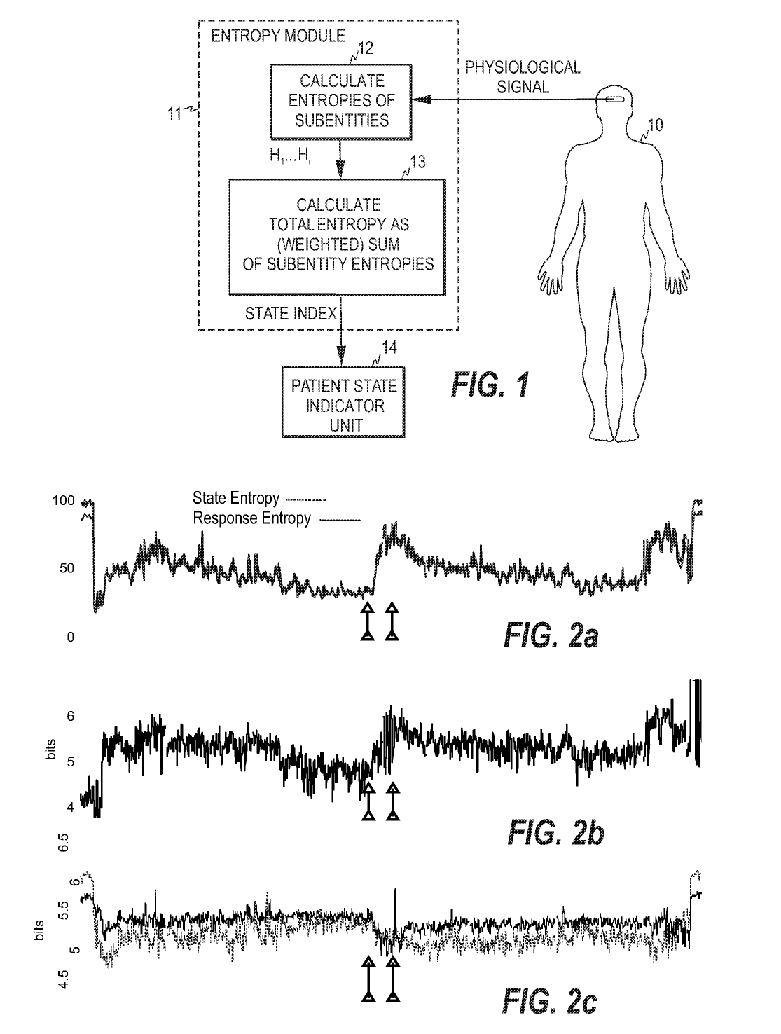

FIG. 1 illustrates the determination of a state index in accordance with one embodiment of the present invention. One or more physiological signals obtained from a subject 10 are supplied to an entropy module 11 in which the entropy of each signal is calculated. It is assumed here that the measurement set-up represents a typical situation in which the physiological signal is an EEG signal and the entropy represents the spectral entropy of the EEG signal.

Entropy analysis has been proved to be useful in analyzing and monitoring of brain wave signals, such as the EEG. Spectral entropy is one of the most widely adapted approaches for estimating the entropy of neuronal activation within the cortex. Neurophysiologists divide the EEG signal to different activities, each corresponding to a dedicated frequency band. The said activities are called delta, theta, alpha, beta and gamma activities, and the respective frequency bands, also called classical frequency bands, are 1-3.5 Hz, 3.5-8 Hz, 8-13 Hz, 13-30 Hz, and 30-70 Hz. These activities are more or less independent of each other and have also different generators within the brain. An EEG signal measured from a subject represents the sum of excitatory and inhibitory potentials of large numbers of cortical pyramidal neurons, which are organized in columns. Each EEG electrode senses the average activity of several thousands of cortical pyramidal neurons.

As is common in the art, the signal obtained from a subject is digitized and the signal data is processed as sets of sequential signal samples representing finite time blocks or time windows, commonly termed “epochs”. The length of the epochs may be fixed or adaptive based on a certain criterion, such as a change in signal stationarity. Also, the epochs may be sliding one or more samples at a time. Furthermore, the signal samples employed do not necessarily correspond to the original measured and digitized samples of the physiological signal, but they may also be processed samples of the signal, such as coefficients and/or samples obtained from a wavelet transform, Fourier transform, or filter bank.

In the entropy module 11 of FIG. 1, the signal samples described above are first decomposed into several signal subentities and the entropy of the each subentity is calculated in a first computing unit 12 of the entropy module. In this example, the subentities correspond to the above-mentioned delta, theta, alpha, beta, and gamma subbands of the EEG signal. In the first computing unit 12, the wideband brain wave signal is thus decomposed into said five consecutive subbands and the entropy of each subband is calculated.

Generally speaking, the first computing unit thus outputs the entropies of the subentities, H₁ . . . Hₖ, where k corresponds to the number of subentities. In this example, the computing unit thus outputs entropies H₁ . . . H₅ corresponding to the entropies of the delta, theta, alpha, beta, and gamma subbands, respectively.

The subband-specific entropy values are supplied to a second computing unit 13, in which an aggregate entropy measure H_{T} representing the total entropy of the signal is calculated. In one embodiment of the invention, the aggregate entropy measure is calculated as the weighted sum of the subentity entropies:

$${H}_{T} = {\sum }_{i = 1}^{k} {w}_{i} \times {H}_{i}$$

wherein the weights wᵢ correspond to the probabilities of the subentities. In the above example, the probability of each subband corresponds to ratio bᵢ/n, where bᵢ is the number of discrete frequency indexes on subband i and n is the total number of discrete frequency indexes on the whole frequency band.

However, generally speaking the sum may be calculated as weighted or unweighted sum of the subentity entropies, i.e., the weights of the subentity entropies may also be equal to each other.

The subband-specific entropy Hᵢ represents the spectral entropy of each band as first presented by Inouye et al.: Quantification of EEG irregularity by use of the entropy of the power spectrum, Electroencephalography and clinical Neurophysiology, 79 (1991), pp. 204-210 (cf. Equation 4 in the article). Inouye et al. were also the first to present spectral entropy over a wide EEG range, such as 1-30 Hz, which covers several different EEG activities, cf. Equation 2 in the article. However, as discussed above, spectral entropy calculated from a frequency range that covers different physiological activities, which may be more or less independent of each other, may behave inconsistently.

As is commonly known in the art, there are various approaches to calculating entropy. Although Shannon entropy is used in the examples illustrated in this specification, other entropy equations, such as Rényi or Tsallis entropy equations, may also be used. As has been demonstrated in the above-mentioned U.S. Pat. No. 6,801,803, for example, complexity measures derived from a time-domain signal resemble spectral entropy, and some of those measures actually originate from entropy theory. Those measures include, for example, approximate entropy and Lempel-Ziv complexity. As obvious for a person skilled in the art, those measures may be utilized for calculating the subband-specific entropies and the resulting aggregate measure, after the EEG signal is first divided into the subbands by suitable filter banks, for example.

In information theory, entropy is commonly defined as the expected value of the information included in the set of random variables. Therefore, entropy can be presented in bits, where the number of bits represents the amount of information contained in the data set. The maximum possible amount of information in a data set of length N is log₂N. For example, in case of spectral entropy calculated from power spectral density of frequency resolution 0.2 Hz, the maximum spectral entropy of the delta band (1.0 Hz . . . 3.4 Hz) corresponds to log₂13, while that of the beta band (13.2 Hz . . . 30.0 Hz) corresponds to log₂85. As is obvious for a person skilled in the art, other base numbers than 2 may be used in the logarithmic operation, although the correspondence to information presented in bits does not hold in that case.

The aggregate entropy measure obtained and presented in bits may directly represent the state index indicative of the level of hypnosis of the subject. The second computing unit may thus output a sequence of the state index, i.e., an aggregate entropy measure for each (sliding or non-sliding) time window of the signal. However, the aggregate entropy measure may also be transformed so that the state index values are more appropriate for the end-user. For example, a transformation may be used to transform the calculated aggregate entropy measures to a relative entropy scale, where the original bit-valued entropy is divided by the maximum possible entropy value. Thus, on the entropy scale will be [0 . . . 1], which may again be transformed to another integer scale, such as [0 . . . 100]. The state index values obtained based on the aggregate entropy measures are shown to the user on the display of a patient state indicator unit 14.

The entropy module 11 of FIG. 1 may be constructed from any computer-based system that is appropriate for determining the total entropy in the above manner. As used herein, the term ‘computer’ may include any processor-based or microprocessor-based system that includes systems using microcontrollers, reduced instruction set circuits (RISC), application-specific integrated circuits (ASIC), logic circuits, and any other circuit or processor that is capable of calculating a measure of the total entropy described herein. The examples given above are exemplary only, and are not intended to limit in any way the definition and/or meaning of the term ‘computer’.

The logarithm of the EEG power spectrum decays almost linearly with increasing frequency, but different activities can be observed from the spectrum as peaks in the corresponding frequency band. These peaks are generated by the synchronous neuronal activity and the spectral entropy within each band is a useful tool for characterizing the activity. However, conventionally spectral entropy is calculated over a wide frequency range, such as 1-32 Hz. Because spectral entropy is calculated over the frequency bins of the power spectrum, such a wide range is sensitive to high amplitude, low frequency activities and mostly neglects the synchronization occurring in the higher frequency bands. Therefore, spectral entropy over the wide frequency range is not fully applicable for the monitoring of neuronal synchronization and, in some cases it merely reflects either the slowing or fastening of EEG rhythms.

By determining the entropy as the sum of the subentity entropies, the neurophysiologic basis of the EEG may be taken into account. The determination takes into account the different classical frequency bands equally, according to information content of each band. A state index determined according to the embodiment of FIG. 1 therefore performs more consistently in connection with drugs that have different effects on different subbands.

Table 1 below presents BIS Index, Response Entropy (RE), and State Entropy (SE) data recorded from 50 subjects totally (n=50). In addition, spectral entropies calculated with traditional method (SpEn trad, as described by the above-mentioned article by Inouye et al.) from the frequency bands 1-32 Hz, 1-47 Hz, and 1-70 Hz are presented. Also, spectral entropies utilizing the sum of subband entropies are presented (SpEn total). Here, the SpEn total 1-70 Hz is calculated from identical time windows as SpEn trad. In addition, SpEn total 1-70 Hz is calculated with cycle compensation, i.e., each subband entropy is estimated from a time window having a length that corresponds to the respective subband frequencies. The data is obtained in connection with slow induction of three anesthetic or sedative drugs: propofol (n=20), sevoflurane (n=10), and dexmedetomidine (n=20). The columns of the table represent the following variables, from left to right: mean of maximum values of the subjects (presenting baseline awake value when no drug is administered), mean of individual values just before loss of consciousness (LOC), mean of individual minimum values (presenting maximal drug effect reached), standard deviation (SD) of values just before LOC, coefficient of variation (CV) of values just before LOC (calculated as the standard deviation divided by the mean value), and standard deviation just before LOC divided by the respective range (where the range is the difference of mean maximum and mean minimum values).

TABLE 1

| | | Mean | | SD | | SD(LOC)/ |
|---|---|---|---|---|---|---|
| | Mean (Max) | (LOC) | Mean (Min) | (LOC) | CV (LOC) | range |
| Propofol, n = 20 | | | | | | |
| BIS | 98.0 | 57.4 | 45.7 | 9.3 | 0.16 | 0.18 |
| RE | 100.0 | 56.8 | 40.2 | 16.1 | 0.28 | 0.27 |
| SE | 90.8 | 52.8 | 34.7 | 13.7 | 0.26 | 0.24 |
| SpEn trad 1-32 Hz | 6.44 | 5.49 | 2.83 | 0.62 | 0.11 | 0.17 |
| SpEn trad 1-47 Hz | 6.90 | 5.55 | 2.96 | 0.63 | 0.11 | 0.16 |
| SpEn trad 1-70 Hz | 7.41 | 5.57 | 3.14 | 0.64 | 0.11 | 0.15 |
| SpEn total 1-70 Hz | 6.08 | 5.52 | 3.88 | 0.17 | 0.03 | 0.08 |
| SpEn total 1-70 Hz, | 4.77 | 4.29 | 3.03 | 0.11 | 0.02 | 0.06 |
| cycle compensation | | | | | | |
| Sevoflurane, n = 10 | | | | | | |
| BIS | 98.0 | 68.4 | 51.7 | 9.1 | 0.13 | 0.20 |
| RE | 100.0 | 74.6 | 44.7 | 17.8 | 0.24 | 0.32 |
| SE | 91.0 | 70.0 | 35.8 | 18.4 | 0.26 | 0.33 |
| SpEn trad 1-32 Hz | 6.49 | 5.86 | 2.84 | 0.37 | 0.06 | 0.10 |
| SpEn trad 1-47 Hz | 6.96 | 5.96 | 2.91 | 0.40 | 0.07 | 0.10 |
| SpEn trad 1-70 Hz | 7.46 | 6.01 | 3.03 | 0.44 | 0.07 | 0.10 |
| SpEn total 1-70 Hz | 6.09 | 5.60 | 3.42 | 0.23 | 0.04 | 0.08 |
| SpEn total 1-70 Hz, | 4.76 | 4.40 | 2.98 | 0.08 | 0.02 | 0.04 |
| cycle compensation | | | | | | |
| Dexmedetomidine, | | | | | | |
| n = 20 | | | | | | |
| BIS | 97.6 | 52.8 | 39.2 | 13.3 | 0.25 | 0.23 |
| RE | 99.8 | 42.2 | 21.9 | 24.7 | 0.58 | 0.32 |
| SE | 90.4 | 37.9 | 19.2 | 20.2 | 0.53 | 0.28 |
| SpEn trad 1-32 Hz | 6.18 | 4.52 | 2.68 | 0.74 | 0.16 | 0.21 |
| SpEn trad 1-47 Hz | 6.67 | 4.57 | 2.81 | 0.78 | 0.17 | 0.20 |
| SpEn trad 1-70 Hz | 7.20 | 4.62 | 2.91 | 0.83 | 0.18 | 0.19 |
| SpEn total 1-70 Hz | 6.11 | 5.72 | 4.06 | 0.15 | 0.03 | 0.07 |
| SpEn total 1-70 Hz, | 4.75 | 4.43 | 3.16 | 0.12 | 0.03 | 0.08 |
| cycle compensation | | | | | | |
| All drugs, n = 50 | | | | | | |
| BIS | 97.8 | 57.7 | 44.3 | 12.35 | 0.21 | 0.23 |
| RE | 99.9 | 54.5 | 33.6 | 23.39 | 0.43 | 0.35 |
| SE | 90.7 | 50.2 | 28.6 | 21.07 | 0.42 | 0.34 |
| SpEn trad 1-32 Hz | 6.35 | 5.17 | 2.77 | 0.84 | 0.16 | 0.23 |
| SpEn trad 1-47 Hz | 6.82 | 5.24 | 2.89 | 0.87 | 0.17 | 0.22 |
| SpEn trad 1-70 Hz | 7.33 | 5.28 | 3.02 | 0.89 | 0.17 | 0.21 |
| SpEn total 1-70 Hz | 6.09 | 5.62 | 3.86 | 0.20 | 0.04 | 0.09 |
| SpEn total 1-70 Hz, | 4.76 | 4.37 | 3.07 | 0.13 | 0.03 | 0.07 |
| cycle compensation | | | | | | |

The SpEn trad and SpEn total values of Table 1 were calculated off-line using the recorded EEG signal data. The spectral entropy values (SpEn) were calculated from time windows of 5 seconds, sliding 1 second at a time. The spectral entropy values so obtained were median filtered with a 9-tap long filter. In case of traditional methods (SpEn trad), entropy was calculated from frequency bands of 1-32 Hz, 1-47 Hz, and 1-70 Hz, using each frequency band in its entirety for the entropy calculation. The sum of the subentity entropies (SpEn total) was calculated as the weighted sum of the spectral entropies in delta (1-3.4 Hz), theta (3.6-8.0 Hz), alpha (8.2-13.0 Hz), beta (13.2-30.0 Hz), and gamma (30.2-48 Hz and 52.0-70.0 Hz) bands. As time windows of 5 seconds were used, the frequency resolution obtained in Fourier transform was 0.2 Hz, which resulted in the following band-specific weights wᵢ: delta 0.0398, theta 0.0703, alpha 0.0765, beta 0.2599, and gamma 0.5535. In this case, weights are probabilities of each subentity.

As can be seen from Table 1, the sum of the subband entropies is superior over traditional spectral entropy in terms of coefficient of variation in LOC and standard deviation of LOC per range, cf. the last two columns of the table. The sum of the subband entropies also behaves clearly better than the commercially available variables. Apart from the sum of subband entropies, all the other methods show much lower values at LOC in case of dexmedetomidine than in case of propofol or sevoflurane. When all subjects are pooled in one group (All drugs, n=50), the solution based on the sum of subband entropies shows an even more remarkable advantage over the other methods, since it is less prone to inter-drug and inter-individual variations.

It should also be noted that it is possible to calculate the spectral entropies of each band from time windows of different lengths. For example, the time window can be chosen to contain approximately 30 cycles of each EEG activity. The spectral entropies of the gamma, beta, alpha, theta, and delta bands may then be calculated, respectively, from time windows of 1, 2, 4, 6, and 24 seconds, for example. Thus, the frequency resolutions of the bands are different, which results in the following band-specific weights (probabilities): 0.3352 (delta), 0.1538 (theta), 0.1154 (alpha), 0.1923 (beta), and 0.2033 (gamma). Also, the maximal information (i.e., entropy) captured by the low frequency bands increases in that case. For example, in this case the maximal entropy for the delta band is log₂60. As presented in Table 1, this kind of cycle compensation reduces the variation even more.

As is demonstrated by this example, the total entropy calculated as a sum of subentity entropies follows the fundamentals of information theory and physics. If longer time windows are used for lower frequencies, more information from that frequency range can be captured. Thus, the estimate contains more information (i.e., the maximum possible entropy value increases). Also, the obtained entropy estimate of the subentity is more reliable, thus more weight may be given for that subentity entropy when calculating the total entropy. Consequently, one possibility is to use criteria related to subentity entropies when selecting the lengths of the time windows for each frequency range.

Ketamine is an analgesic and a hypnotic drug, which induces loss of synchronization in the alpha and beta bands, but increases the synchronization in the gamma band. The net synchronization effect of ketamine is positive (entropy decreases), because gamma activation cancels out the deactivations of alpha and beta bands. FIG. 2a to 2c illustrate the behavior of different entropies during a surgery when ketamine is administered. FIG. 2a illustrates the State Entropy (SE) and Response Entropy (RE) calculated similarly as in the above-mentioned commercial S/5 Entropy Module. FIG. 2b illustrates traditional spectral entropy calculated from a frequency range of 1-70 Hz, and FIG. 2c illustrates spectral entropy calculated in the above manner as the sum of subband entropies from two frequency bands: 1-30 Hz (dashed line) and 1-70 Hz (solid line). In the figures, an anesthesiologist has first administered ketamine (first arrow). This causes an increase in the spectral entropy calculated traditionally, cf. FIGS. 2a and 2b, which the anesthesiologist falsely interprets to represent too light anesthesia. The anesthesiologist therefore administers more sevoflurane (second arrow). However, the total spectral entropy calculated as the sum of subband entropies behaves logically, as can be seen from FIG. 2c. The total entropy decreases after the ketamine bolus and remains lower at the second half of the recording where sevoflurane level is higher than in the first half.

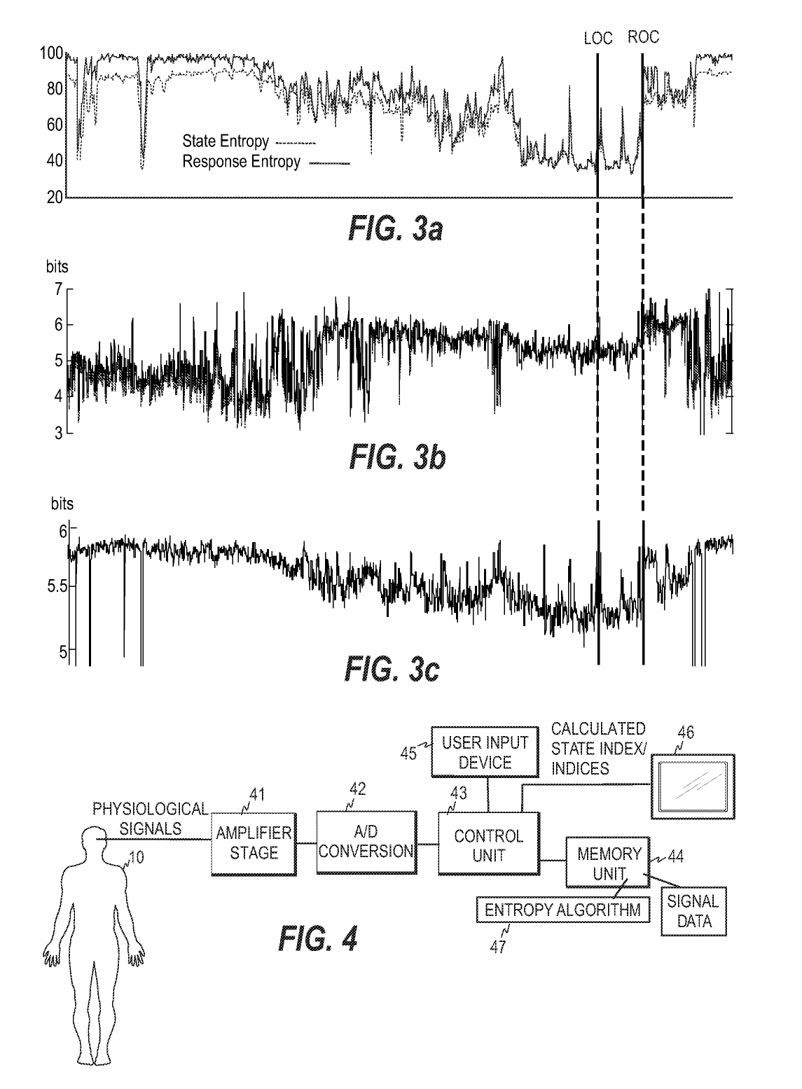

FIG. 3a to 3c illustrates an example of spectral entropies obtained in connection with slow propofol administration. FIG. 3a illustrates the State and Response Entropies, SE and RE. FIG. 3b shows spectral entropy values calculated according to the traditional method from frequency ranges 1-32 Hz, 1-47 Hz, and 1-70 Hz. FIG. 3c illustrates total spectral entropy from the range 1-70 Hz calculated as the sum of the entropies of the above 5 subbands. The vertical lines shown in the figures present time of loss of consciousness (LOC) and return of consciousness (ROC). As can be seen, spectral entropy calculated according to the traditional method is prone to eye movement artifacts, which cause low spectral entropy values in awake subject moving his/her eyes. In contrast, spectral entropy calculated as the sum of subband entropies remains high also during the eye movement periods.

FIG. 4 illustrates one embodiment of the system or apparatus according to the invention. The physiological signal(s) obtained from one or more sensors attached to a subject 10 are supplied to an amplifier stage 41, which amplifies the signal(s) before they are sampled and converted into digitized format in an A/D converter 42. The digitized signals are supplied to a control unit 43 which may comprise one or more processor-based or microprocessor-based systems. As discussed above, the signal data measured from the subject is typically brain wave signal data, which is measured through electrodes applied to the forehead of the subject. The electrodes normally also receive EMG signal data resulting from the activity of the facial muscles. Instead of EEG data, magnetoencephalographic (MEG) signal data may also be measured. MEG is indicative of the magnetic component of brain activity, i.e., it is the magnetic counterpart of EEG.

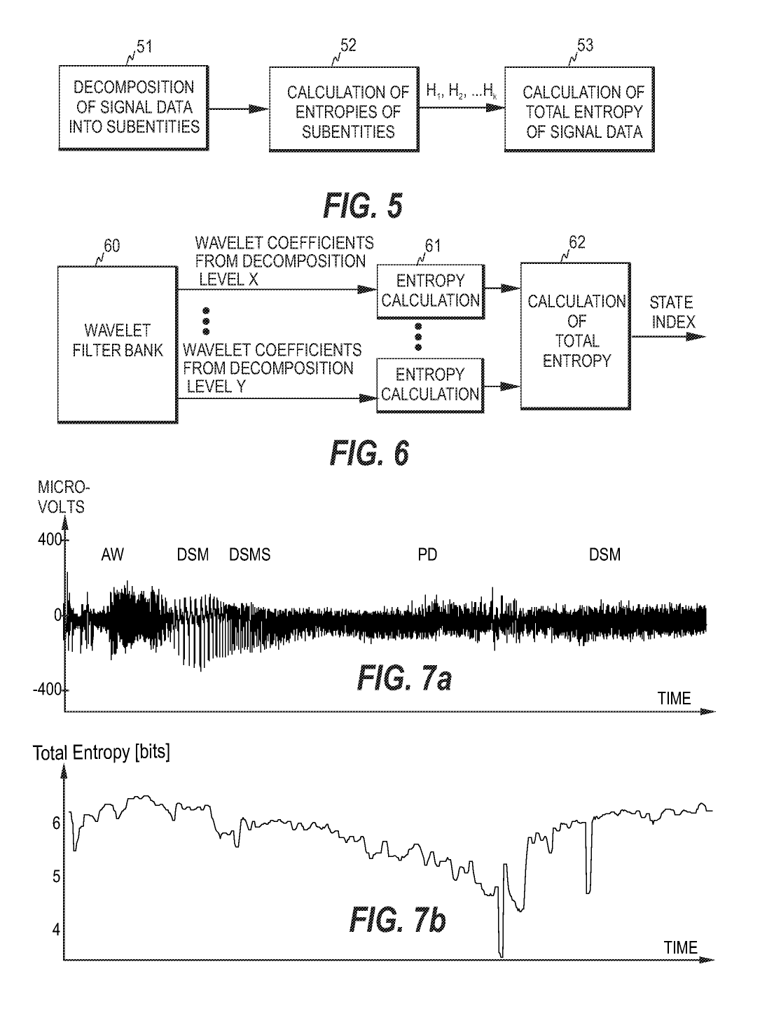

The computer unit is provided with a memory or database 44 holding the digitized signal data obtained from the sensor(s). The memory or database may also store an entropy algorithm 47 used for determining an aggregate entropy measure indicative of the total entropy and/or an entropy-based state index based on the aggregate entropy measure. The memory may further comprise algorithm(s) for one more other state indices that may possibly be determined based on the physiological data obtained from the subject. The control unit, which is equipped with the above entropy algorithm, may be seen as an entity of three consecutive operational modules or units, as is illustrated in FIG. 5: a decomposing unit 51 configured to decompose the signal data of one or more physiological signals into desired subentities, a calculation unit 52 configured to calculate the entropies of the subentities, and a total entropy calculation unit 53 configured to calculate, based on the entropies of the subentities, the aggregate entropy measure representing the total entropy. The aggregate entropy measure or a value derived from the said measure is supplied to a monitor 46. The user of the apparatus/system controls the operation of the apparatus/system through one or more user input devices 45 that form, together with the monitor, the user interface of the apparatus/system. Through the user interface the user may define various parameters related to the method, such as the widths and/or weights of the subbands.

Although one computer unit or processor may perform the steps of the invention, the processing of the data may also be distributed among different units/processors (servers) within a network, such as a hospital LAN (local area network). The apparatus of the invention may thus also be implemented as a distributed system.

A conventional patient monitor may also be upgraded to enable the monitor to determine entropy according to the invention. Such an upgrade may be implemented by delivering to the patient monitor a software module that enables the device to calculate entropy or a state index in the above-described manner. The software thus comprises the algorithm 47 in the form of program code that can be executed by the control unit. The software module may be delivered, for example, on a data carrier, such as a CD or a memory card, or through a telecommunications network. The upgrade may replace the old entropy algorithm of the patient monitor or the monitor may be provided with a new state index that is based on the sum of the subentity entropies.

Instead of the sum of the subentity entropies, any other measure that behaves similarly as the sum may be employed as the aggregate entropy measure. For example, a product of the subentity entropies might serve as the aggregate entropy measure indicative of the total entropy.

As discussed above, in one embodiment of the invention a set of predefined signal waveforms may represent the signal subentities. In order to detect specific epileptiform patterns, it has been suggested to decompose the EEG signal by a wavelet filter bank and to calculate the entropies of the wavelet coefficients for the desired wavelet scales of the filter bank. This results in several subband-specific entropy values, each value being indicative of presence of respective waveforms in the original EEG signal. Each value thus represents the entropy of the coefficients indicative of the presence of the waveform defined by the respective scale and the mother wavelet. FIG. 6 illustrates an embodiment in which the subentity-based entropy calculation is applied to this kind of detection of epileptiform activity. The wavelet coefficients are output from desired decomposition levels of a wavelet filter bank 60. The wavelet coefficients are squared to power of two in order to obtain the energy distribution of the signal in time-domain and within each scale. The entropy of each energy distribution is calculated in a subentity entropy unit 61, thereby to obtain a plurality of simultaneous subentity entropy values. The total entropy of the energy distribution of the original time-domain signal is then calculated as the weighted or unweighted sum of the subentity entropies in an entropy unit 62, which outputs the state index. Here, the signal subentities can thus be regarded to correspond to the waveforms defined by the selected decomposition levels (scales) and the mother wavelet of the transform, since the coefficients output from a certain decomposition level are indicative of the presence of waveforms defined by that decomposition level (scale) and the mother wavelet used. As in previous examples, the subentities are typically chosen so that they cover substantially the entire frequency range of the original EEG signal.

FIGS. 7a and 7b show an example of the behavior of the state index of FIG. 6 when an EEG signal includes epileptiform activity. FIG. 7a shows the original EEG signal measured from a subject, while FIG. 7b shows the total entropy calculated from the said EEG signal as the weighted sum of five subentity entropies. The EEG patterns shown in FIG. 7a are as follows: awake (AW), delta slow monophasic (DSM), delta slow monophasic with spikes (DSMS), and periodic epileptiform discharges (PD).

FIG. 7b presents the total entropy of energy distributions in time over all scales, calculated as the weighted sum of the energy distribution entropies from the scales corresponding to frequency ranges 32-64 Hz, 16-32 Hz, 8-16 Hz, 4-8 Hz, and 2-4 Hz. In this example, Mallat algorithm and Daubechies 3 mother wavelet was employed and the use of a sample frequency of 128 Hz and a time window of 5 seconds resulted in scale-specific weights of 0.5, 0.25, 0.125, 0.0625, and 0.0313, respectively. As can be seen from FIG. 7b, the total entropy calculated as the weighted sum of subentity entropies decreases monotonically with increasing severity of epileptiform activity in the EEG signal. Thus, the sum of subentity entropies may be employed as a state index indicative of epileptiform activity in the subject.

Although the number of the subentities is typically chosen so that the subentities cover substantially the entire frequency range of the physiological signal, the number of subentities may also depend on the application and/or the measurement equipment used. For example, the delta frequencies may not be necessary when epileptiform activity is detected, while the gamma frequencies may be left out for the reason that some of the existing EEG measurement devices do not record the said higher EEG frequencies.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural or operational elements that do not differ from the literal language of the claims, or if they have structural or operational elements with insubstantial differences from the literal language of the claims.

1. A method for monitoring physiological state of a subject, the method comprising:
obtaining physiological signal data from a subject;
decomposing the physiological signal data into a plurality of signal subentities;
determining, for each of the signal subentities, a first measure indicative of the entropy of respective signal subentity, thereby to obtain a corresponding plurality of first measures;
calculating an aggregate entropy measure from the plurality of first measures, the aggregate entropy measure being indicative of total entropy of the physiological signal data; and
employing the aggregate entropy measure to produce at least one state index indicative of a physiological state of the subject.

2. The method according to claim 1, wherein the decomposing includes decomposing the physiological signal data into the plurality of signal subentities, wherein the signal subentities comprise subbands of an overall frequency range of the physiological signal data.

3. The method according to claim 2, wherein the decomposing includes decomposing the physiological signal data into the plurality of subbands, wherein the subbands are consecutive subbands of the overall frequency range of the physiological signal data.

4. The method according to claim 3, wherein the calculating includes calculating the aggregate entropy measure and wherein the physiological signal data comprises EEG signal data and the subbands comprise delta, theta, alpha, beta, and gamma subbands of the EEG signal data.

5. The method according to claim 1, wherein the calculating includes calculating the aggregate entropy measure, wherein the aggregate entropy measure is indicative of a sum of the plurality of the first measures.

6. The method according to claim 5, wherein the calculating includes calculating the sum and wherein the sum is a weighted sum of the plurality of the first measures.

7. The method according to claim 6, wherein the calculating includes calculating the weighted sum and wherein the weighted sum comprises weights that correspond to probabilities of the signal subentities.

8. The method according to claim 1, wherein the decomposing includes decomposing the physiological signal data into the plurality of signal subentities, wherein the decomposing includes producing a plurality of wavelet coefficient sets, wherein each set represents a respective signal subentity of the plurality of signal subentities.

9. The method according to claim 1, wherein the employing includes using the aggregate entropy measure directly as the state index.

10. The method according to claim 1, wherein the employing includes transforming the aggregate entropy measure to the state index, wherein the state index is within a predetermined index scale.

11. An apparatus for monitoring physiological state of a subject, the apparatus comprising:
a signal decomposer configured to decompose physiological signal data obtained from a subject into a plurality of signal subentities;
a determination unit configured to determine, for each of the signal subentities, a first measure indicative of the entropy of respective signal subentity, thereby to obtain a corresponding plurality of first measures;
a first calculation unit configured to calculate an aggregate entropy measure from the plurality of first measures, the aggregate entropy measure being indicative of total entropy of the physiological signal data; and
a second calculation unit configured to employ the aggregate entropy measure to produce at least one state index indicative of a physiological state of the subject.

12. The apparatus according to claim 11, wherein the signal subentities comprise subbands of an overall frequency range of the physiological signal data.

13. The apparatus according to claim 12, wherein the subbands are consecutive subbands of the overall frequency range of the physiological signal data.

14. The apparatus according to claim 13, wherein the physiological signal data comprises EEG signal data and wherein the subbands comprise delta, theta, alpha, beta, and gamma subbands of the EEG signal data.

15. The apparatus according to claim 11, wherein the aggregate entropy measure represents a sum of the plurality of the first measures.

16. The apparatus according to claim 15, wherein the sum is a weighted sum of the plurality of the first measures.

17. The apparatus according to claim 11, wherein the plurality of signal subentities is represented by a corresponding plurality of wavelet coefficient sets output from a wavelet filter.

18. The apparatus according to claim 11, wherein the second calculation unit is configured to use the aggregate entropy measure directly as the state index.

19. The apparatus according to claim 18, wherein the second calculation unit is configured to transform the aggregate entropy measure to the state index, wherein the state index is within a predetermined index scale.

20. A computer program product for an apparatus monitoring a subject, the computer product comprising:
a first program code portion configured to decompose physiological signal data obtained from a subject into a plurality of signal subentities;
a second program code portion configured to determine, for each of the signal subentities, a first measure indicative of the entropy of respective signal subentity, thereby to obtain a corresponding plurality of first measures;
a third program code portion configured to calculate an aggregate entropy measure from the plurality of first measures, the aggregate entropy measure being indicative of total entropy of the physiological signal data; and
a fourth program code portion configured to employ the aggregate entropy measure to produce at least one state index indicative of a physiological state of the subject.
